(19) United States
(12) Patent Application Publication
(10) Pub. No.: US 2022/0062450 A1
(43) Pub. Date: Mar. 3, 2022

(54) PORTABLE HEATING STERILIZATION STORAGE BAG FOR FACE MASKS AND CLOTH PRODUCTS

(71) Applicants: Li Chieh Cheng, New Taipei City (TW); Yi-Yan Yang, New Taipei City (TW)

(72) Inventors: Li Chieh Cheng, New Taipei City (TW); Yi-Yan Yang, New Taipei City (TW)

(21) Appl. No.: 17/008,620

(22) Filed: Aug. 31, 2020

(51) Int. Cl. A61L 2/00

(52) U.S. Cl. CPC A61L 2/0023; A61L 2202/16; A61L 2202/21; A61L 2202/122

(57) ABSTRACT

A portable heating sterilization bag for face mask and cloth products includes a bag container. The bag is capable of holding and containing face masks. At least one of heating plate installed on one inner surface in the bag and being made of god conductor material of heat; a heat source is for heating the at least one heating plate; the heat source is attached to the at least one heating plate and serving to control the internal temperature of the bag. If the power switch is turned on, the heat plate heats the contained face mask or cloth product to produce the effect of face mask or cloth product disinfect and sterilization. The heating plate is made of metal or non-metal material and it can be rigid or flexible.

### FIELD OF THE INVENTION

The present invention is related to sewing fabric masks or disposable non-woven medical masks, and in particular to a portable heating sterilization storage bag for face masks and cloth products.

### BACKGROUND OF THE INVENTION

Face Masks are used to filter bacteria, viruses, and dusts in the air. The face masks cover on the mouths and noses of users so as to isolate their mouths and noses from the most of harmful materials in the environment. If the face masks are not in use, they are contained in bags, boxes, or packets of clothes for storage. Cloth products also exists same problem occurred in the face masks.

However, when a face mask or a cloth product is used through a certain time period, or it is placed in a highly polluted environment, the face mask or cloth product is easy to be polluted with dusts, bacteria, virus, or other harmful materials on it. As this kind of result, users must take a new one to replace the polluted one to maintain the protection effect. As a result, users must prepare many face masks or cloth product in storage. Not only the cost of preparing heating sterilization pot is high, but also many wasted face masks or cloth products can cause many environmental protection problems. If they are not well processed, the wasted face masks or cloth products will become infection sources in the environment and are harmful to the mankind.

Therefore, the inventors of the present invention desire to develop a novel design which is portable and with the sterilization function of face mask and cloth products at any time when its user wants. Furthermore, it can extend the lifecycle of the face mask and cloth products to be longer. This is beneficial for individual face mask and cloth products sterilization, especially, this is beneficial for face mask and cloth products sterilization while user traveling. As a result, the lifecycle of the face mask and cloth products are prolonged and the cost compared to the preparation of face masks or other sterilization machine is also less.

### SUMMARY OF THE INVENTION

Accordingly, for improving above mentioned defects in the prior art, the object of the present invention is to provide a portable heating sterilization storage bag for face mask and cloth products, wherein when a user places a face mask or a cloth product on the heating plate, the heating plate produces the effect of face mask or cloth product disinfect and sterilization. And this heating plate can be either rigid or flexible. Thus, the user can put on a clean face mask or a cloth product without biological pollution and the lifecycles of them are prolonged after heating by this bag. As a result, the original face mask and the cloth product can be repeatedly used in many times.

To achieve above object, the present invention provides a portable heating sterilization storage bag for face mask and cloth products which includes a bag; at least one of heating plate installed on one inner surface of the bag and being made of good conductor material of heat; a heat source for heating the at least one heating plate; the heat source being attached to the at least one heating plate and serving to control the internal temperature of the bag. The bag is capable of holding and containing face masks or cloth products. If the power switch is turned on, the heating plate heats the contained face mask or clothe product to produce the effect of face mask or cloth product disinfect and sterilization. The heating plate is made of metal or non-metal material and can be rigid or flexible.

In one application, there is only one heating plate. The outer surface of the heating plate is attached with an additional smaller clamping plate to hold the face mask or the cloth product; the heat source can be attached to the inner surface of the heating plate or the outer surface of the smaller clamping plate and serves to control the internal temperature of the bag.

Furthermore, in another application, there are two heating plates; each heating plate connects with each other along one of their lateral sides to form a t bag; the outer surface of each heating plate is attached with a respective smaller clamping plate; each clamping plate belongs to the inner surface structure of the bag; at least one heat source is attached to the at least one heating plate and serve to control the internal temperature of the bag.

### BRIEF DESCRIPTION OF THE DRAWINGS

FIG. 1 is an assembled schematic view showing the components in the first embodiment of the present invention.

FIG. 2 is a schematic view showing the application of the first embodiment of the present invention.

FIG. 3 is an assembled structural schematic view showing the assembly of the components of the first embodiment of the present invention.

FIG. 4 is an assembled schematic view showing the components in the second embodiment of the present invention.

FIG. 5 is a schematic view showing the application of the second embodiment of the present invention.

FIG. 6 is an assembled structural schematic view showing the assembly of the components of the second embodiment of the present invention.

FIG. 7 is an assembled schematic view showing the components in the third embodiment of the present invention.

FIG. 8 is a schematic view showing the application of the third embodiment of the present invention.

FIG. 9 is a schematic view showing the wallet of the present invention according to the third embodiment of the present invention, which is in a storage state.

FIG. 10 is another schematic view showing the wallet of the present invention according to the third embodiment of the present invention,

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

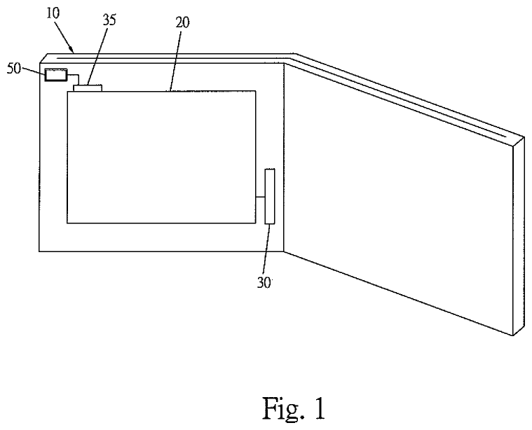

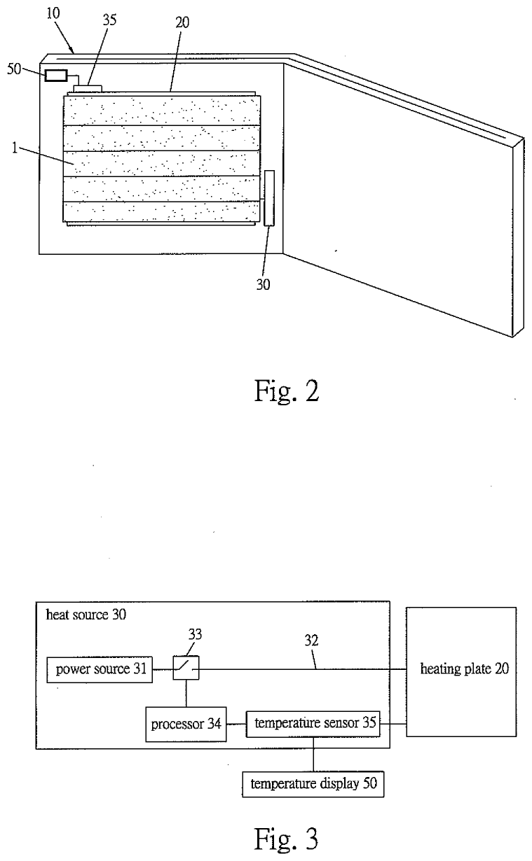

With reference to FIGS. 1 to 3, the structure of the present invention is illustrated. The present invention related to a portable heating sterilization storage bag for face masks and cloth products. The bag of the present invention is suitable for sterilize face masks or cloth products. In the following, the fact masks are used in the description, but it does not used to confine the scope of the present invention. As shown in the title of the invention, the bag of the present invention is suitable for face masks or cloth products includes the following elements.

A bag 10 is a portable device for storage.

At least of heating plate 20 is installed at the inner surface of the bag and is made of good conductor material of heat1, such as heat conductor metals, or non-metal materials.

A heat source 30 serves to heat the at least one heating plate 20. The heat source 30 is connected to the at least one heating plate 20 and serves to control the device temperature of the at least one heating plate 20 so that the temperature of the heating plate 20 is between 56° C. and 70° C. and the temperature is retained through a period. The temperature of 56° C. is over a temperature for condensing protein and thus the property of the protein is changed.

A heat source 30 contains the following elements.

A power source 31 is connected to the heating plate 30 through conductive wires 32 for heating the at least one heating plate 20.

A switch 33 is arranged on the conductive wire 32 for controlling conduction of the conductive wire 32.

A processor 34 is connected to the switch 33 for controlling turning one or off of the switch 33.

A temperature sensor 35 is arranged on the heating plate 20 and serves to transfer detected temperatures to the processor 34. When the temperature is lower than 56° C., the processor 34 will actuate the power source 31 for heating the heating plate 20. When the temperature is higher than 70° C., the processor 34 will turn off the power source 31 to stop heating of the heating plate 20.

Other than controlled by the processor 34, the switch 33 can be turned on or off manually.

In application, a face mask 1 (or a cloth product, in drawings of the present invention, face masks are depicted and are used in the description of the invention, however this is not used to confine the scope of the present invention) is placed into the bag 10 and is adhered on the heating plate 20. At this moment, a user could switch on the switch 33 to heat the heating plate 20 to have the effect of disinfect and sterilization. When the face mask 1 is not used, it is placed in the bag 20. The processor 34 serves to control temperatures of the heating plate 20 so that the temperature of the heating sheet 20 is between 56° C. and 70° C. to retain the face mask 1 in a sterilizable state so as to achieve the objects of safety use of the face mask.

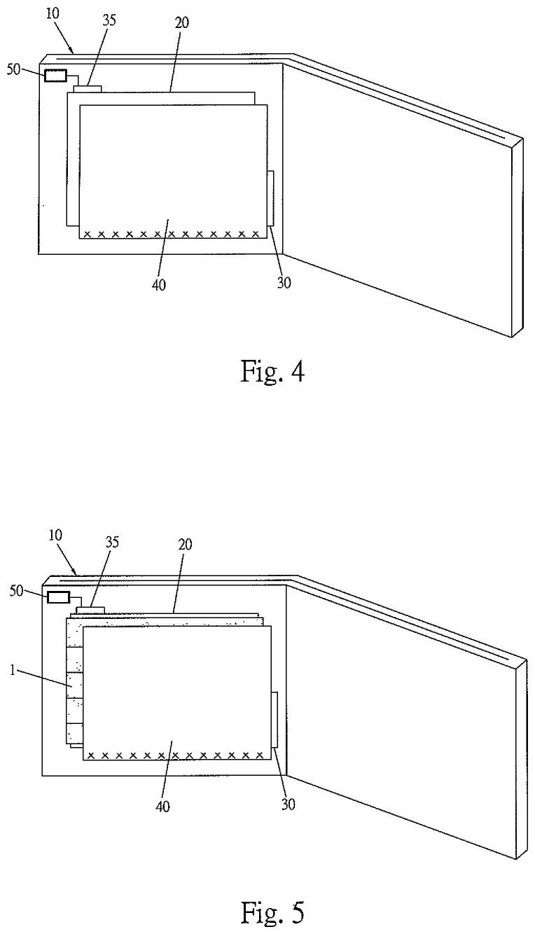

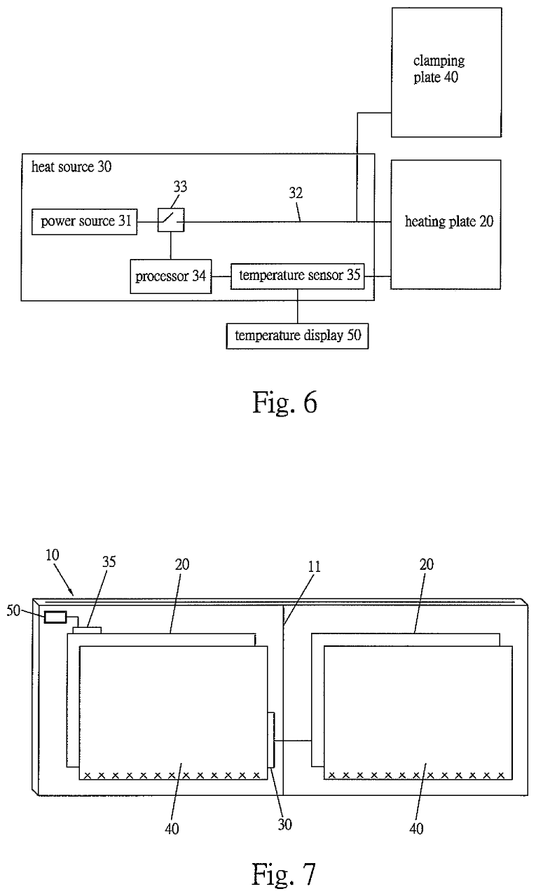

With reference to FIGS. 4 to 6, the second embodiment of the present invention is illustrated. In this application, the elements identical to the above embodiment have identical function and thus will not be further described herein.

However, in this embodiment, an outer surface of the heating plate 20 is attached with an additional clamping plate 40 which is fixed to the bag 20 by any desire way, such as it is glued on an inner surface of the bag 20 and covers on an upper side of the clamping plate 40. The heat source 30 is connected to the clamping plate 40 and serves to control the device temperature of the clamping plate 40 to be between 56° C. and 70° C. When the temperature detected by the temperature sensor 35 is lower than 56° C., the processor 34 will actuate the power source 31 for heating the heating plate 20 and the clamping plate 40. When the temperature is higher than 70° C., the processor 34 will turn off the power source 31 to stop heating of the heating plate 20 and the clamping plate 40.

In application, a face mask 1 is placed into the bag 10 and is between the heating plate 20 and clamping plate 40. At this moment, a user could switch one the switch 33 so as to heat the heating plate 20. Thereby, the face mask 1 is heated from two sides to have preferred heating effect.

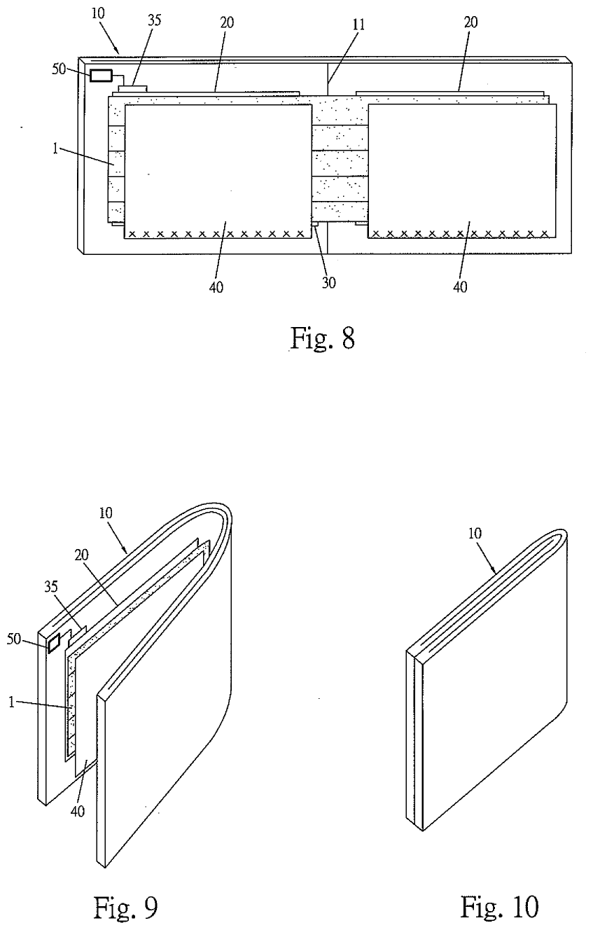

With reference to FIGS. 7 to 10, the third embodiment of the present invention is illustrated. In this application, the elements identical to the above embodiment have identical function and thus will not be further described herein.

In this embodiment, the at least one heating plate 20 is two heating plates 20. Each heating plate 20 is adhere on two lateral sides of the bag 10. An outer surface of each heating plate 20 is attached with a respective clamping plate 40. Each clamping plate 40 is fixed to the inner surface of the bag 10 by any way, for example, by gluing on the inner surface of the bag 10 and covers on the heating plate 20. The heat source 30 is also connected to the clamping plate 40 and serves to control the device temperature of the clamping plate 40 to be between 56° C. to 70° C. When the temperature detected by the temperature sensor 35 is lower than 56° C., the processor 34 will actuate the power source 31 for heating the heating plate 20 and the clamping plate 40. When the temperature detected by the temperature sensor 35 is higher than 70° C., the processor 34 will turn off the power source 31 to stop heating of the heating plate 20 and the clamping plate 40.

In application, a face mask 1 is placed flatly between the heating plate and the clamping plate 40. Then the bag 10 is folded along a middle folded line to reduce the whole volume thereof. and thus the bag 10 could be placed in a pocket of clothes. FIG. 10 shows that a bag 10 is in a folded state. In use, the user 1 could open the switch 33 so that heating plates 20 and the clamping plates 40 heat the face mask 1 to achieve the effect of disinfect and sterilization. When the face mask 1 is unused, it is placed within the bag 10. The processor 34 will control the device temperature of the heating plates 20 and the clamping plates 40 to be between 56° C. to 70° C. so as to retain the face mask 1 in a sterilizable state and to achieve the object of safety use.

The main object of the third embodiment is that in generally, the volume of a face mask 1 is larger. If the face mask 1 is unfolded, it cannot be placed in the packet. Therefore by folded bags, the face masks 1 can be placed in the bag and thus they can be placed in a pocket to be portable easily and conveniently.

In all mentioned embodiments above, the bag 10 could be installed with a temperature display 50 which is connected to the temperature sensor 35. The temperature display 50 serves to display the temperatures detected by the temperature sensor 35 so that the user may view of the temperature anytime.

Advantages of the present invention are that when a user places a face mask on the heating plate, the heating plate has the effect of disinfect and sterilization of the face mask. The user can wear a clean face mask without pollution and thus lifetime of the face mask is prolonged. As a result, the original face mask can be reused many times.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A portable heating sterilization storage bag for face mask and cloth products, comprising:
a bag;
at least of one heating plate installed on one inner surface of the bag and being made of good conductor material of heat;
a heat source for heating the at least one heating plate; the heat source being attached to the at least one heating plate and serving to control the internal temperature of the bag; and
wherein the bag is capable of holding and containing at least a face mask or a cloth product; if the power switch is turned on, the heating plate heats the contained face mask or the cloth product to produce the effect of face mask or cloth product disinfect and sterilization.

2. The portable heating sterilization storage bag for face masks and cloth products as claimed in claim 1, wherein the heat plate is made of metal or non-metal material and is rigid or flexible.

3. The portable heating sterilization storage bag for face mask and cloth products as claimed in claim 1, wherein the heat source comprises:
an power source connected to the heating plate through conductive wires for heating the at least one heating plate;
a switch arranged on the conductive wire for controlling conduction of the conductive wire;
a processor connected to the switch for controlling turning one and off of the switch; and
a temperature sensor arranged on the heating plate for transferring detected temperatures to the processor based on the temperature transferred from the temperature sensor, wherein the processor actuates the switch so as to cause the heat source to heat the heating plate or turn off the switch so as to stop the heat source and thus not to heat the heating plate.

4. The portable heating sterilization storage bag for face mask and cloth products as claimed in claim 1, wherein the heat source controls the internal temperature of the device to be between 56° C. and 70° C.; when the temperature is lower than 56° C., the processor actuates the power source for heating the heating plate; when the temperature is higher than 70° C., the processor turns off the power source to stop heating.

5. The portable heating sterilization storage bag for face mask and cloth products as claimed in claim 1, wherein other than controlled by the processor, the power switch is turned on or off manually.

6. The portable heating sterilization storage bag for face mask and cloth products as claimed in claim 1, wherein an outer surface of the heating plate is attached with a clamping plate which is attached to the heat source and
wherein in application, a face mask or a cloth product is placed into the bag and is between the heating plate and the clamping plate; when the power switch is on, the heating plate is heated; and thereby, the face mask or the cloth product is heated.

7. The portable heating sterilization storage bag for face mask and cloth products as claimed in claim 6, wherein the heat source serves to control the internal temperature of the device to be between 56° C. and 70° C.; when the temperature detected by the temperature sensor is lower than 56° C., the processor actuates the power source for heating; when the temperature is higher than 70° C., the processor turns off the power source to stop heating.

8. The portable heating sterilization storage bag for face mask and cloth-products as claimed in claim 1, wherein the at least one heating plate is two heating plates; each heating plate is connect to each other along one of their lateral sides to form a the bag; an outer surface of each heating plate is attached with a respective clamping plate; each clamping plate belongs to the inner surface structure of the bag; the heat source is also connected to the clamping plates and serves to control the internal temperature of the device; and
wherein in application, a face mask or a cloth product is placed between the heating plate and the clamping plate; then the bag is folded along a middle folded line to reduce the whole volume thereof and thus the bag could be placed in a pocket of clothes; the switch serves to control the device to heat the face mask or the cloth product to achieve an effect of face mask disinfect and sterilization.

9. The portable heating sterilization storage bag for face mask and cloth products as claimed in claim 8, wherein the heat source serves to control the internal temperature of the bag to be between 56° C. and 70° C.; when the temperature detected by the temperature sensor is lower than 56° C., the processor actuates the power source for heating the heating plates and the clamping plates; when the temperature detected by the temperature sensor is higher than 70° C., the processor turns off the power source to stop heating.

10. The portable heating sterilization storage bag for face mask and cloth products as claimed in claim 1, wherein the bag is installed with a temperature display which is connected to the temperature sensor; and the temperature display serves to display the temperatures detected by the temperature sensor; the temperature display is attached on an outer surface of the bag or is connected with the bag by a wire cable.
